(19) United States
(12) Patent Application Publication
(10) Pub. No.: US 2009/0283050 A1
(43) Pub. Date: Nov. 19, 2009

(54) FEED COMPOSITION AND METHOD FOR BREEDING ANIMALS

(75) Inventors: Satomi Tsuchibe, Meguro-ku (JP); Toshiyasu Takada, Kashiwa-shi (JP); Masashi Uchida, Komaki-shi (JP); Yasuo Kurihara, Nagoya-shi (JP)

Correspondence Address: OBLON, SPIVAK, MCCLELLAND MAIER & NEUSTADT, L.L.P., 1940 DUKE STREET, ALEXANDRIA, VA, 22314, US

(73) Assignees: KENKO CORPORATION, Tokyo (JP); SINANEN ZEOMIC CO., LTD., Nagoya-Shi (JP)

(21) Appl. No.: 12/469,289

(22) Filed: May 20, 2009

Related U.S. Application Data
(62) Division of application No. 11/133,274, filed on May 20, 2005, now abandoned.

(30) Foreign Application Priority Data
Mar. 25, 2005 (JP) 2005-89310

(51) Int. Cl. A01K 67/00

(52) U.S. Cl. 119/174

(57) ABSTRACT

The present invention relates to a feed composition which contains silver-carrying zeolite and an animal-breeding method comprising the step of administering, to an animal, the foregoing silver-carrying zeolite-containing feed composition. The feed composition has almost no influence on the useful lactobacillus, but exerts an antimicrobial effect on various kinds of harmful microorganisms, in particular, bacteria belonging to the genus Salmonella or the like. Accordingly, the feed composition would permit the improvement of the healthiness of enteric canal of domestic animals, domestic fowls and cultivated fishes and the composition would in thus permit the acceleration of their growth, while maintaining their good healthiness.

### CROSS-REFERENCE TO RELATED APPLICATIONS

The present application is a divisional of U.S. patent application Ser. No. 11/133,274, filed on May 20, 2005, which claims priority to Japanese patent application JP 2005-089310, filed on Mar. 25, 2005.

### BACKGROUND OF THE INVENTION

The present invention relates to a feed composition for use in, for instance, poultry (domestic fowls) and livestock (domestic animals) such as chickens, cattle and swine and a method for breeding an animal using the feed composition, and more specifically to a feed composition which has almost no influence upon the useful lactobacillus, but exerts a strong antimicrobial effect on various kinds of harmful microorganisms, in particular, bacteria belonging to the genus Salmonella or the like, as well as a method for breeding an animal using such a feed composition.

The consuming public has recently been concerned about the safety of livestock and aquatic products or foods such as edible meat, eggs, and fishes and shellfishes. In this respect, there have been known a variety of harmful microorganisms such as bacteria and viruses which can invade the bodies of cattle, poultry and cultivated fishes and settle in the bodies of these animals. As a result, individuals may be infected with such various kinds of microorganisms through, for instance, the intake of a food contaminated with the same, in particular, bacteria belonging to the genus Salmonella and this in turn results in the occurrence of various hygienic injuries such as food poisoning and a variety of serious diseases and this accordingly becomes an object of public concern.

There has not yet been completely elucidated a route through which various harmful microorganisms such as bacteria and viruses, in particular, bacteria belonging to the genus Salmonella causing these food poisoning and serious diseases enter into human and animal bodies. In this respect, it has in general been recognized that, in most cases, these microorganisms may enter into the bodies through various feeds or foods and/or drinking water, as mediators, ingested by these animals. In this connection, as methods for protecting animals from being poisoned by foods or from being infected with the foregoing causal bacteria or viruses for the serious diseases described above, there may be listed, for instance, a method for sterilizing feeds or foods and drinking water; or a method for imparting, to these animals, an antimicrobial activity which permits the control of any proliferation of such harmful microorganisms, for instance, pathogenic bacteria.

There has conventionally been proposed a feed composition to which an antimicrobial activity is imparted by the incorporation, into the feed composition, of a variety of antimicrobial agents such as antibiotics, sulfa drugs and iodine-containing compounds (see, for instance, Patent Document No. 1 specified below). However, these antimicrobial agents may suffer from various problems such that they show unstable antimicrobial effects on highly resistive bacteria such as those belonging to the genus Salmonella and if they enter into human bodies through the intake of a food derived from an animal bred using the antibacterial agent-containing feed composition, they may induce allergic reactions in the human bodies and/or may exert an adverse effect on the valuable intestinal bacteria such as lactobacillus.

### Patent Document No. 1: JP-A-2003-40717.

### SUMMARY OF THE INVENTION

Accordingly, it is in general an object of the present invention to provide a feed composition containing an antimicrobial agent and a method for breeding an animal while making use of the feed composition.

The present invention provides a feed composition and a method for breeding animals using the same as detailed hereinbelow.

### BRIEF DESCRIPTION OF THE DRAWINGS

FIG. 1 is a flow diagram representing an administration schedule of a feed composition comprising a silver-carrying zeolite in accordance with an exemplary breeding method of the present invention.

FIG. 2 is a flow diagram representing an administration schedule of a feed composition comprising a silver-carrying zeolite in accordance with an exemplary breeding method of the present invention.

FIG. 3 is a flow diagram representing an administration schedule of a feed composition comprising a silver-carrying zeolite in accordance with an exemplary breeding method of the present invention.

FIG. 4 is a flow diagram representing an administration schedule of a feed composition comprising a silver-carrying zeolite in accordance with an exemplary breeding method of the present invention.

### DETAILED DESCRIPTION OF THE INVENTION

According to the first aspect of the present invention, there is provided a feed composition for breeding animals characterized in that it contains silver-carrying zeolite.

In a preferred embodiment of the foregoing feed composition, the amount of silver contained in the silver-carrying zeolite ranges from 0.1 to 5.0% by mass on the basis of the total mass of the silver-carrying zeolite.

In another preferred embodiment of the foregoing feed composition, the content of silver in the composition ranges from 0.000001 to 0.2% by mass (0.01 to 2000 ppm) on the basis of the total mass of the feed composition.

In still another preferred embodiment of the foregoing feed composition, the amount of silver contained in the silver-carrying zeolite ranges from 0.1 to 5.0% by mass on the basis of the total mass of the silver-carrying zeolite and the content of silver in the feed composition ranges from 0.000001 to 0.2% by mass (0.01 to 2000 ppm) on the basis of the total mass of the composition.

According to the second aspect of the present invention, there is provided a method for breeding an animal which comprises the step of administering, to an animal, a feed composition containing silver-carrying zeolite.

In a preferred embodiment of the foregoing breeding method, the amount of silver contained in the silver-carrying zeolite ranges from 0.1 to 5.0% by mass on the basis of the total mass of the silver-carrying zeolite.

In another preferred embodiment of the foregoing breeding method, the content of silver in the feed composition ranges from 0.000001 to 0.2% by mass (0.01 to 2000 ppm) on the basis of the total mass of the composition.

In still another preferred embodiment of the foregoing breeding method, the amount of silver contained in the silver-carrying zeolite ranges from 0.1 to 5.0% by mass on the basis of the total mass of the silver-carrying zeolite and the content of silver in the feed composition ranges from 0.000001 to 0.2% by mass (0.01 to 2000 ppm) on the basis of the total mass of the composition.

According to a further preferred embodiment of the foregoing breeding method, the feed composition having a silver content of A % by mass (A being not less than 0.000001) is administered to the animal in the initial breeding (Starter) stage.

According to a still further preferred embodiment of the foregoing breeding method, the feed composition having a silver content of A % by mass (A being not less than 0.000001) is administered to the animal in the initial breeding (Starter) stage and the composition having a silver content of B % by mass (B<A) is administered to the animal after the completion of the initial breeding (Starter) stage.

In a still further preferred embodiment of the foregoing breeding method, the animal is a layer or milk cow.

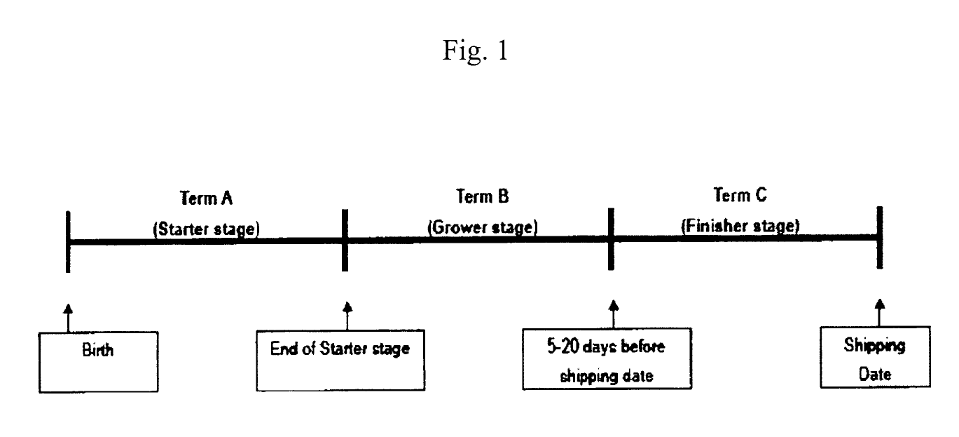

In a still further preferred embodiment of the foregoing breeding method, the composition having a silver content of A % by mass (A being not less than 0.000001) is administered to the animal in the initial breeding (Starter) stage; the composition having a silver content of B % by mass (B<A) is administered to the animal during the term between the end of Starter stage and 5 to 20 days before a shipping date of animals; and the composition having a silver content of C % by mass (C>B) is administered to the animal during the term between a shipping date and 5 to 20 days before the shipping date, as shown in FIG. 1.

According to a further preferred embodiment of the foregoing breeding method, the animal is one for obtaining meat.

According to a still further preferred embodiment of the foregoing breeding method, the animal is poultry, cattle, swine, a wild boar, an equine, sheep or a deer.

According to a further preferred embodiment of the foregoing breeding method, the animal is a domestic fowl for obtaining chicken, beef cattle or swine.

The feed composition of the present invention has almost no influence upon the useful lactobacillus, but can exert a substantial or strong antimicrobial effect on various kinds of harmful microorganisms, in particular, bacteria belonging to the genus Salmonella or the like. Accordingly, the feed composition would permit the improvement of the healthiness of enteric canal of domestic animals, domestic fowls and cultivated fishes such as chickens, swine, cattle, equines, carps and various kinds of cultivated fishes and the composition would, in turn, permit the acceleration of their growth. Moreover, the meat, milk, eggs and other livestock products derived from the animals bred by administering the feed composition of the present invention are free of any contamination with a variety of harmful microorganisms, in particular, bacteria belonging to, for instance, the genus Salmonella and therefore, they are quite safe from the viewpoint of the food sanitation.

Further, in the method of the present invention, the composition having a silver content of A % by mass (A being not less than 0.000001) is administered to the animal in Starter stage or during the term extending from its birth to its childhood until the animal acquires the immunity almost comparable to that of the adult thereof and therefore, the method of the invention would permit the prevention from any infection of the animal in its childhood with various harmful microorganisms.

Furthermore, in the method of the present invention, the composition having a silver content of A % by mass (A being not less than 0.000001) is administered to the animal in Starter stage and the composition having a silver content of B % by mass (B<A) is administered thereto after the completion of Starter stage. Accordingly, the method of the invention permits the efficient use of expensive silver and the effective breeding of animals while maintaining the healthy conditions thereof and ensuring a high breeding efficiency. In particular, when breeding layers with the feed composition of the present invention, the fowls would provide eggs over a quite long period of time.

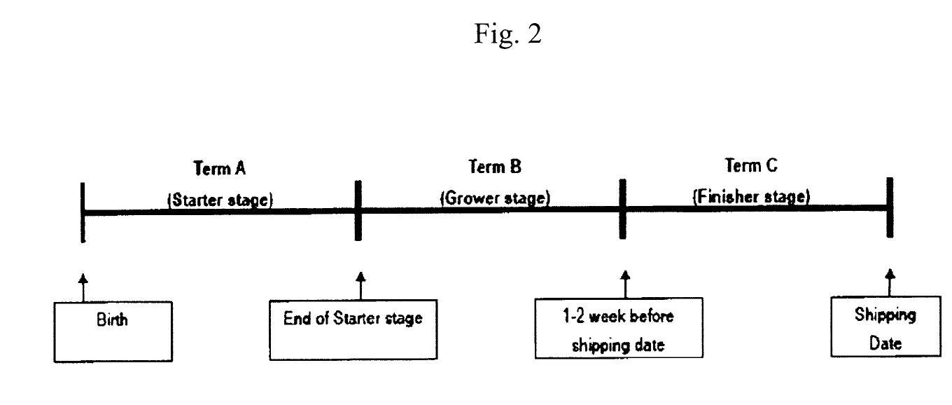

In addition, in a preferred embodiment of the method according to the present invention, the composition having a silver content of A % by mass (A being not less than 0.000001) is administered to the animal in Starter stage; the composition having a silver content of B % by mass (B<A) is administered to the animal during the term between the end of Starter stage and about one to two weeks before a shipping date of the animal; and the composition having a silver content of C % by mass (C>B) is then administered to the animal during the term between the shipping date and about one to two weeks before the shipping date of the animal, as shown in FIG. 2. Accordingly, the method of the invention permits the efficient use of expensive silver and the effective breeding of animals, in particular, those bred for obtaining meat, while maintaining the healthy conditions of the animals and ensuring a high breeding efficiency.

If using the feed composition of the present invention, it is expected that animals can be protected from the infection with, for instance, pathogenic bacteria without using any antibiotics at all and this may in turn inhibit the generation of any resistant bacteria possibly generated due to frequent use of antibiotics and this likewise permits the complete protection of human bodies from the infection with such pathogenic bacteria or the reduction of the probability that individuals are infected with the bacteria.

Further, the feed composition of the present invention has almost no bad influence on bacteria useful for animals such as lactobacillus, but exerts a high antimicrobial effect on various kinds of harmful microorganisms, in particular, bacteria belonging to, for instance, the genus Salmonella and accordingly, the composition would permit the maintenance of well balanced intestinal flora. As a result, the offensive smells of the animal's excretion can significantly be reduced, this in turn permits the reduction of any deterioration of the environment around the facilities for breeding animals due to the generation of such bad smells. Moreover, the foregoing would permit the foundation of such facilities for breeding animals in areas in the proximity to cities and this may likewise result in the achievement of such an effect that the cost required for the transportation of livestock products can considerably be reduced.

### BEST MODE FOR CARRYING OUT THE INVENTION

The present invention will hereunder be described in more detail.

The term “silver-carrying zeolite” as used in the present invention means zeolite carrying silver having antimicrobial activity in its stable form. In this connection, examples of such zeolite usable herein include naturally occurring products such as zeolite A, zeolite X, zeolite Y, zeolite T, zeolite having a high silica content, sodalite, mordenite, analcime, clinoptilolite, chabazite and erionite; semi-synthesized products called artificial zeolite; and chemically synthesized products.

Examples of antimicrobial agents usable herein as carriers for silver also include silver-containing glass, zirconium silver phosphate, silver-containing silica gel, silver-containing apatite, fine particles of silver, protein-silver, sulfur diazine-silver, in addition to zeolite type ones such as those listed above, but the use of zeolite type ones is preferred in the present invention because they can ensure the achievement of a stable antimicrobial activity.

Then, a method for preparing such a silver-carrying zeolite product will be described below in detail.

The silver-carrying zeolite used in the present invention can be prepared according to a wet or dry technique. The wet technique comprises, for instance, following steps: zeolite is introduced into a silver ion-containing solution prepared in advance to thus allow the occurrence of ion exchange between silver ions and those content in the zeolite and exchangeable with the former in the liquid phase. This ion-exchange may be carried out according to the batch-wise or continuous method (for instance, a column method) and the operation may be conducted at a temperature ranging from 10 to 70° C. and preferably 40 to 60° C., for 3 to 32 hours and preferably 10 to 24 hours. In this respect, it is suitable to adjust the pH value of the silver ion-containing solution to a level ranging from 3 to 10 and preferably 5 to 7. The silver ion-containing solution used herein is in general prepared from any silver salt such as silver nitrate, silver sulfate, silver perchlorate, silver acetate, diammine silver nitrate and diammine silver sulfate.

The amount of the silver ions to be applied to or deposited on zeolite can be adjusted by controlling the silver ion concentration in the silver ion-containing solution used in the foregoing ion-exchange reaction. When using a silver ion-containing solution having a silver ion concentration falling within the range of from 0.005 to 0.15 M/L, various silver-carrying zeolite products can appropriately be prepared, which have a desired silver ion content ranging from 0.1 to 5.0% by mass on the basis of the total mass of the silver-carrying zeolite. In this connection, the silver ion-containing solution used in the ion-exchange reaction with zeolite may likewise contain cations other than silver ions such as zinc ions, copper ions, manganese ions, nickel ions, magnesium ions, calcium ions and/or ammonium ions in order to improve the chemical stability of the resulting silver-carrying zeolite. Various kinds of these additional cations can easily be applied to zeolite by the addition of these cations to the silver ion-containing solution. In this respect, it is in general suitable that the various kinds of cations other than silver ions to be applied onto or deposited on the zeolite is controlled to the range of from 0 to about 4 moles per unit mole of silver ions.

On the other hand, the dry technique usable herein may comprise, for instance, the steps of mixing zeolite particles with silver salt particles and then heat-treating the resulting mixture to remove the anions contained in the silver salt and to thus give silver-carrying zeolite particles. Alternatively, a silver salt solution prepared by dissolving the silver salt in a small amount of water can be substituted for the silver salt particles used in the foregoing dry technique to thus give silver-carrying zeolite particles. In this connection, the heat-treatment may suitably be carried out at a temperature ranging from 200 to 700° C. and preferably 400 to 600° C., for 20 to 120 minutes and preferably about 30 to 50 minutes. Silver salts usable in the dry technique may be, for instance, those listed above in connection with the wet technique. Similarly, zeolite particles carrying cations other than silver ions together with the latter can be prepared by the use of an aqueous solution containing a salt other than a silver salt in addition to the latter, like the foregoing wet technique.

In the present invention, the amount of silver supported by or included in the zeolite preferably ranges from 0.1 to 5.0% by mass and more preferably 0.5 to 2.5% by mass on the basis of the total mass of the silver-carrying zeolite, from the viewpoints of the antibiotic activity against the bacteria or the like present in animal bodies and the safety thereof.

The amount of the silver-carrying zeolite particles to be incorporated into the feed composition of the present invention preferably ranges from 0.001 to 0.2% by mass and more preferably 0.01 to 0.1% by mass on the basis of the total mass of the resulting feed composition.

Moreover, the feed composition of the present invention may likewise be prepared in the form of a concentrated feed composition (Premix) and the latter may be diluted with the usual feed composition such that the resulting mixture has a silver content specified above before administering the same to animals. The silver-carrying zeolite content in the concentrated feed composition preferably ranges from 0.1 to 10% by mass and more preferably 0.5 to 5% by mass on the basis of the total mass of the concentrated feed composition (Premix).

The feed composition of the present invention may further contain optional components such as a various kinds of amino acids, minerals, enzymes and/or lactobacillus, in addition to the foregoing components for the supplementation of nutrients, the nutritional enrichment, the improvement of digestion and absorption, the promotion of growth and the impartment of disease resistance.

The feed composition of the present invention can easily be prepared by admixing powdery raw materials in a mixer. In this respect, the particle size of each raw material is not restricted to any particular level and it can, if necessary, be adjusted while taking into consideration each particular application or the kinds of animals to be bred.

The present invention further provides a method for breeding animals characterized by administering a feed composition containing silver-carrying zeolite particles.

The animals to be bred according to the method of the present invention are not restricted to specific ones.

The animals suitable for the breeding method of the present invention may be, for instance, a variety of animals bred or cultivated as, for instance, livestock, poultry and pet animals and specific examples thereof include mammals, birds, fishes, reptiles and batrachians. The animals to which the feed composition of the present invention can suitably be administered include, for instance, various valuable substances such as meat, eggs, milk, hair, furs, feathers, down, fells and/or skins.

Examples of animals for obtaining meat are animals and fowls such as chickens, cattle, swine, goats, equines, sheep, deer, bears, reindeer, rabbits, ostriches, domestic ducks, turkeys and quails; and cultivated fishes such as trout, eels and young yellowtails.

Examples of animals for egg collection are birds and fishes listed above in connection with the animals for obtaining meat.

Examples of animals for collecting milk usable herein are cows, she-goats and equines.

Examples of animals for collecting hairs (or fur, feathers or downs), furs or skins include cattle, equines, swine, sheep, goats, deer, bears, reindeer, dogs, cats, rabbits, minks, martens, foxes, raccoon dogs and birds for collecting feathers and/or downs.

The animals to which the feed composition of the present invention can suitably be administered include, for instance, animals for obtaining meat and those for egg collection among the animals specifically listed above and specific examples thereof are domestic animals and domestic fowls such as chickens for obtaining meat (chicken), beef cattle, cattle for collecting milk and meat, swine, goats, equines, rabbits, ostriches, domestic ducks, turkeys and quails and, in particular, chickens for obtaining meat (chicken), chickens for egg collection, beef cattle, swine and ostriches.

Further, when the feed composition according to the present invention is administered to pet animals such as small birds and small animals; aquarium fishes such as carps, goldfishes and tropical fishes; laboratory animals such as rats and mice; and various kinds of animals kept in zoological gardens, these animals can be protected beforehand from the possible infection with any infectious pathogenic bacteria and/or viruses.

The animal-breeding method of the present invention is characterized in that a feed composition is administered to each animal in such a manner that the content of the silver-carrying zeolite is changed depending on the different growth periods of the animal and more specifically, the method is characterized in that a feed composition having a relatively high content of silver-carrying zeolite is administered to an animal during the term between its birth and its childhood until the animal acquires the immunity almost comparable to that of the adult thereof (i.e., “Starter stage” or “term A”) during which the animal has only a low resistance to exogenous pathogenic bacteria and/or viruses (hereunder also simply referred to as “resistance to pathogenic bacteria”). This would accordingly permit the considerable reduction in morbidity rate of these animals having only low resistance to pathogenic bacteria and generate the healthy growth of these animals up to their adults within a very short period of time. More specifically, the method of the invention is characterized in that a feed composition having a silver content of A % by mass (A being not less than 0.000001) is administered to an animal in its Starter stage. For instance, when using silver-carrying zeolite particles whose silver content is 2.5% by mass, a feed composition having a silver-carrying zeolite content of 0.001% by mass is administered to an animal in its Starter stage. The silver-carrying zeolite content in a feed composition may vary depending on, for instance, the silver content of each specific silver-carrying zeolite used and the kinds of animals to be bred with the composition and the applications of these animals thus bred, but when using silver-carrying zeolite particles having a silver content of 2.5% by mass, the silver-carrying zeolite content in the feed composition is usually not less than 0.001% by mass (or not less than 10 ppm), preferably not less than 0.005% by mass (or not less than 50 ppm), more preferably not less than 0.01% by mass (or not less than 100 ppm) and most preferably not less than 0.02% by mass (or not less than 200 ppm). The upper limit of the silver-carrying zeolite content in the feed composition of the invention is not restricted to any particular value. However, the use or administration of the silver-carrying zeolite particles in an unreasonably high content would not be desirable from the economical standpoint and it may adversely affect the animals to which the feed composition is administered. Accordingly, the content of the silver-carrying zeolite particles in the feed composition of the invention is suitably not more than 0.1% by mass (or 1000 ppm) based on the total mass of the same. In case where the silver content of the silver-carrying zeolite is other than 2.5% by mass, the silver content in the feed composition will be adjusted appropriately in such a manner that it falls within the range specified above.

The foregoing “Starter stage” or “term A” may vary depending on the kinds of animals to be bred with the feed composition and the applications of these animals thus bred. For instance, in case of layers, it corresponds to 16 to 20 weeks after the hatching and preferably about 3 to 5 weeks after the hatching; in case of domestic fowls for obtaining chicken (broiler), it corresponds to 18 to 25 days after the hatching and preferably about 14 to 21 days after the hatching; in case of swine, it corresponds to the term elapsed from its birth to the instance at which the body weight thereof reaches at least 30% by mass and preferably at least about 25% by mass on the basis of the body weight of the adult thereof, for instance, if the body weight of the adult thereof is 120 kg, the term required till the body weight reaches at least 36 kg and preferably at least about 30 kg; in case of beef cattle, it corresponds to the term elapsed from its birth to the instance at which the body weight thereof reaches at least 38% by mass and preferably at least about 25% by mass on the basis of the body weight of the adult thereof, for instance, if the body weight of the adult of beef cattle is 800 kg, it corresponds to the term required till the body weight reaches at least about 300 kg and preferably at least about 200 kg.

In a preferred embodiment of the breeding method according to the present invention, a composition having a silver content of A % by mass (A being not less than 0.000001) is administered to an animal in its Starter stage and a composition having a silver content of B % by mass (B<A) is administered to the animal after the completion of Starter stage. For instance, when using silver-carrying zeolite particles having a silver content of 2.5% by mass, a composition having a silver-carrying zeolite content of not less than 0.001% by mass is administered to an animal in its Starter stage and a composition having a silver-carrying zeolite content of less than 0.001% by mass is administered to the animal during a desired term after the completion of Starter stage (hereunder also referred to as “term B”).

The silver content in the feed composition administered to an animal during the term B may vary depending on the kinds of animals to be bred with the composition and the applications of these animals thus bred, but it would be sufficient that the silver content satisfies the following relation: [the silver content in the composition administered during the term A]>[the silver content in the composition administered during the term B]. In this respect, when using silver-carrying zeolite particles having a silver content of 2.5% by mass, the feed composition is usually administered in an amount of less than 0.02% by mass (less than 200 ppm) as expressed in terms of the amount of the silver-carrying zeolite, while the lower limit is not particularly specified and B may be equal to zero. In general, however, the dose of the silver-carrying zeolite preferably ranges from 0.0001 to 0.05% by mass (1 to 500 ppm) and more preferably 0.001 to 0.01% by mass (10 to 100 ppm) as expressed in terms of the amount of the silver-carrying zeolite incorporated into the feed composition. In case where the silver content of the silver-carrying zeolite is other than 2.5% by mass, the silver content in the feed composition will be adjusted appropriately in such a manner that it falls within the range specified above.

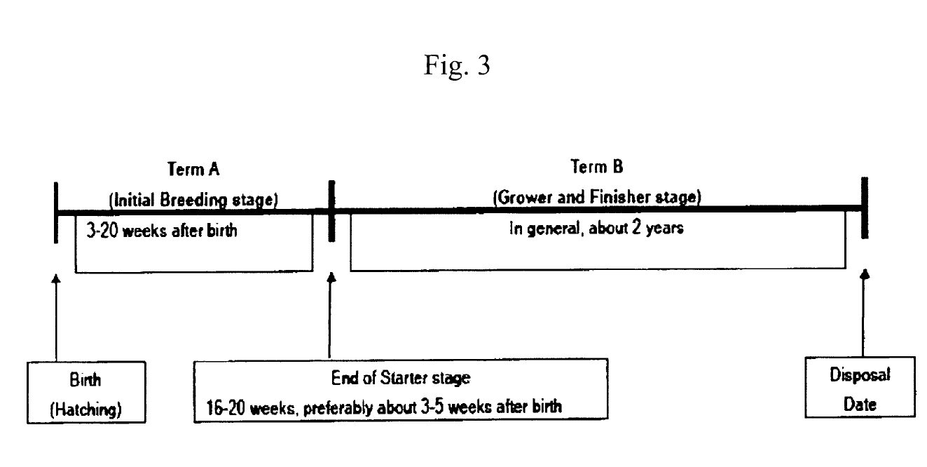

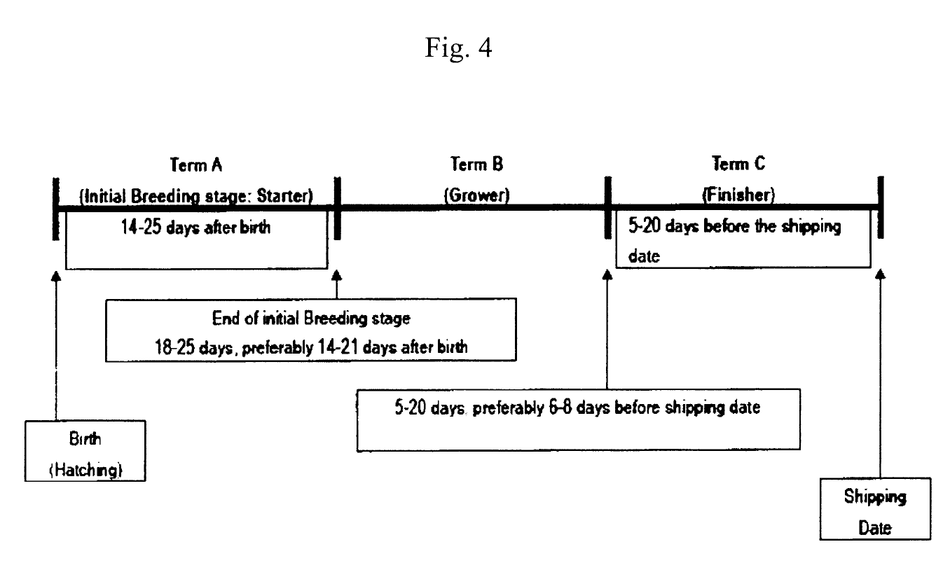

The foregoing “term B”, which corresponds to the desired term after the completion of Starter stage, may vary depending on the kinds of animals to be bred with the feed composition and the applications of these animals thus bred. For instance, in case of layer, it corresponds to the term from the end of the term A namely about 16 to 20 weeks after the hatching and preferably about 3 to 5 weeks after the hatching, up to the disposal of chickens (in general about 2 years), as shown in FIG. 3; in case of broiler, it corresponds to the term between the end of the Starter stage: 18 to 25 days after the hatching and preferably about 14 to 21 days after the hatching and the day that is preferably 5 to 20 days before the shipping date and more preferably 6 to 8 days before the shipping date, as shown in FIG. 4; in case of beef cattle and swine, it corresponds to the term between the end of the term A and the day that is preferably 5 to 20 days before the shipping date and more preferably 6 to 14 days before the shipping date.

In a further preferred embodiment of the breeding method of the present invention, a feed composition having a silver content of A % by mass (A being not less than 0.000001) is administered to an animal in Starter stage of the animal (during the term A); a feed composition having a silver content of B % by mass (B<A) is administered to the animal during the term between the end of Starter stage and the day that is preferably 5 to 20 days before the shipping date; and a feed composition having a silver content of C % by mass (C>B) is administered to the animal during the term between the shipping date and 5 to 20 days before the shipping date. For instance, when using silver-carrying zeolite particles having a silver content of 2.5% by mass, the feed composition having a silver-carrying zeolite content of not less than 0.001% by mass is administered to the animal in Starter stage thereof; the feed composition having a silver-carrying zeolite content of less than 0.001% by mass is administered to the animal during the term between the end of Starter stage and the day that is 5 to 20 days before the shipping date (or during the term B); and the feed composition having a silver-carrying zeolite content of not less than 0.001% by mass is administered to the animal during the term between the shipping date and 5 to 20 days before the shipping date (hereunder also referred to as “term C”).

The silver content in the feed composition administered to an animal during the term C may vary depending on the kinds of animals to be bred with the composition and the applications of these animals thus bred, but it would be sufficient that the silver content satisfies the following relation: [the silver content in the composition administered during the term C]>[the silver content in the composition administered during the term B]. In this respect, when using silver-carrying zeolite particles having a silver content of 2.5% by mass, the feed composition is usually administered in an amount of not less than 0.001% by mass (not less than 10 ppm), preferably not less than 0.005% by mass (not less than 50 ppm), more preferably not less than 0.01% by mass (not less than 100 ppm) and most preferably not less than 0.02% by mass (not less than 200 ppm) as expressed in terms of the amount of the silver-carrying zeolite, while the upper limit of the silver content in the feed composition administered during the term C is not particularly specified. However, the use or administration of the silver-carrying zeolite particles with unreasonably high dosage would be less economical and it may adversely affect the animals. Accordingly, when using silver-carrying zeolite particles having a silver content of 2.5% by mass, the content of silver-carrying zeolite should be not more than 0.1% by mass (or not more than 1000 ppm) as expressed in terms of the amount of the silver-carrying zeolite incorporated into the composition. In case where the silver content of the silver-carrying zeolite is other than 2.5% by mass, the silver content in the feed composition will be adjusted appropriately in such a manner that it falls within the range specified above.

The silver content A may be equal to the silver content C and in this case, the feed composition having the same silver-carrying zeolite content can be used during the both terms A and C.

For instance, in case of broiler, the term A (the initial breeding [Starter] stage) corresponds to the term up to 21 days after its birth; the term B (grower stage) is from 22 to 35 days after the birth; and the term C (finisher stage) is from the end of the term B to the shipping date.

In case of beef cattle, the term A (the initial breeding [Starter] stage) corresponds to the term until when the body weight becomes 400 kg but in general not more than about 300 kg; the term B (grower stage) corresponds to the term between 300 kg and 600 kg in body weight; and the term C (finisher stage) corresponds to the term after the body weight of the animal becomes more than 500 kg but in general when it becomes between 600 and 700 kg.

In case of swine, the term A (the initial breeding [Starter] stage) corresponds to the term until when the body weight becomes 30 kg; the term B (grower stage) corresponds to the term between 20 kg and 70 kg in body weight but usually between 30 kg and 60 kg; and the term C (finisher stage) corresponds to the term when the body weight is between 60 kg and 120 kg.

The animal-breeding method of the present invention is characterized by dividing the growth period of each animal into the following three terms: the term A (the initial breeding [Starter] stage), the term B (grower stage) and the term C (finisher stage), and administering, to the animal, feed compositions having different silver-carrying zeolite contents depending on the different growth or breeding terms. In other words, a feed composition having a high silver-carrying zeolite is administered to an animal in the term during which the animal has a relatively low immunity and resistance to pathogenic bacteria to thus prevent from the infection of the animal with pathogenic bacteria such as those belonging to the genus Salmonella; a feed composition having a lowest possible amount of the silver content is administered to the animal during the term wherein the animal acquires sufficient immunity and high resistance to pathogenic bacteria as a result of sufficient growth to thus prevent the infection of the animal with any pathogenic bacteria economically efficiently and to promote and/or maintain the healthy growth and activity of the animal. However it does not necessarily follow that the dosage and method of administration at each period is different from others.

The present invention will hereunder be described in more detail with reference to the following non-limitative working Examples, but the present invention is not restricted to these specific Examples at all.

### Preparation Example No. 1

### Preparation of Silver-Carrying Zeolite and Feed Composition Containing the Same

Silver-carrying zeolite particles were prepared as follows: There was added water to 1 kg of powder obtained by drying commercially available Zeolite A (Na₂O.Al₂O₃.2SiO₂.xH₂O; average particle size: 1.5 μm) at 110° C. to thus give 1.3 L of a slurry and then the slurry was stirred at 20° C. for 18 hours. Appropriate amounts of a nitric acid aqueous solution and water were added to the slurry to control the pH value thereof to a level ranging from 5 to 7. Then the slurry was poured into 3 L of a 0.075 M/L aqueous solution of silver nitrate and the resulting mixture was continuously stirred at 50° C. for 24 hours to thus establish an equilibrium condition. After the completion of the reaction, the zeolite phase was filtered off and washed with water to remove, for instance, excess silver ions. The resulting zeolite phase was dried at 110° C. to thus give silver-carrying zeolite particles. The silver content of the resulting zeolite particles was found to be 2.5% by mass.

Thereafter there were prepared various kinds of feed compositions containing the resulting silver-carrying zeolite particles incorporated therein. Examples of the formulations thereof are summarized in the following Table 1. In this respect, there were also prepared comparative samples which were free of any silver-carrying zeolite particles or to which Ampicillin as a typical antibiotic agent was incorporated.

### Test Example No. 1

Confirmation test on the Antimicrobial Effect of Feed Composition contains silver-carrying zeolite particles prepared as per the above Preparation Example No. 1 by administering it to broiler chicken.

Test was carried out using broiler chicken for the confirmation of the antimicrobial effects of the feed compositions which contain silver-carrying zeolite particles prepared as per the above Preparation Example No. 1 and the details of feed compositions are listed in Table 1. In the following, the term “part” means “part by mass” unless otherwise specified.

These tests were carried out using 5 test groups (including 1,400 broiler chickens in all groups; each group corresponds to Tests 1 to 5) each comprising 7 test subgroups (comprising 280 broiler chickens) wherein each subgroup consisted of 40 broiler chickens including hens and cocks (20 broiler chickens each). The experimental result for each test group was defined to be the average of the corresponding 7 subgroups wherein the average value for each subgroup corresponded to the average value of the broiler chickens constituting each subgroup. In these tests, the term A (the initial breeding [Starter] stage) was defined to be the term between 0 and 21 days after the hatching of these chicks; the term B (grower stage) was defined to be the term between 22 and 35 days after the hatching thereof, and the term C (finisher stage) was defined to be the term between 36 and 42 days after the hatching thereof and these test were completed after 42 days from the hatching thereof. The following Table 2 shows the details of the feed compositions administered to every test group during every corresponding growth period.

The broiler chickens in each test group were evaluated by inspecting them for the following various characteristic properties: the rate (%) of body weight gain, the feed conversion ratio, lesion score, and the plate count of Salmonella found in the feces of each chicken. The results thus obtained are summarized in the following Table 3.

Body Weight Gain (g): This is herein defined as the number obtained by subtracting the initial body weight from the body weight at the end of the test.
Feed Conversion Ratio: This is defined as the value that feed consumption divided by body weight gain.
Lesion Score This is an index for evaluating the healthiness of the enteric canal of broiler chicken. More specifically, this is evaluated by observing the surface of the enteric canal under fluorescent lighting as well as by dissecting the enteric canal to examine the contents and to observe the inside of enteric canal on any affect of, for example, E. tenella (coccidia). These evaluations are done by veterinarian according to the following criteria for E. tenella as an example and the scores are averaged:

0: There is no symptom of any lesion;

+1: There are a few scattered petechiae, but there is no thickening of the cecal wall. The cecal contents are usually normal.

+2: There is the presence of some blood and petechiae which are apparent on the serosal surface are somewhat more numerous. The cecal wall is slightly thickened.

+3: There is more severe bleeding or cecal core (clotting and lumps in grayish cheese like or banana shape) in cecum. Marked thickening of the cecal wall, and deformation and contraction of cecum are clearly observed.

+4: The cecum is considerably contracted and the lesion reaches even the rectum area. The cecal wall is extremely thickened and the blood clotting or cecal core is observed in cecum. In this respect, if the lesions are not uniform on the both sides of the cecum, the evaluation thereof should be carried out on the basis of more severe one.

Number of Salmonella Present in Feces: This was evaluated according to the following criteria:

High Rate of Risk (++): Not less than 10³ cfu/g; It would be highly possible that the meat of broiler chicken is contaminated with Salmonella and it in turn becomes a cause of various defects from the viewpoint of the food sanitation.

Some Risk May be Present (+): Not less than 10² cfu/g; There would be such a possibility that the meat of the broiler chicken is contaminated with Salmonella and it in turn becomes a cause of defects from the viewpoint of the food sanitation.

Almost No Risk (−): Less than 10² cfu/g; There is hardly any such a possibility that the product is contaminated and it in turn becomes a cause of defects from the viewpoint of the food sanitation.

TABLE 1

| Feed | Main Component of Feed | Antibiotic Component | Other Additives |
|---|---|---|---|
| Z1 | Corn/Soybean: | Silver-Carrying Zeolite: | Amino acids, minerals: |
| | 100 parts | 0.01 part | 4 parts |
| Z2 | Corn/Soybean: | Silver-Carrying Zeolite: | Amino acids, minerals: |
| | 100 parts | 0.02 part | 4 parts |
| Z3 | Corn/Soybean: | Silver-Carrying Zeolite: | Amino acids, minerals: |
| | 100 parts | 0.04 part | 4 parts |
| Z4 | Corn/Soybean: | Silver-Carrying Zeolite: | Amino acids, minerals: |
| | 100 parts | 0.08 part | 4 parts |
| N1 | Corn/Soybean: | None | Amino acids, minerals: |
| | 100 parts | | 4 parts |
| N2 | Corn/Soybean: | Antibiotic Agent: | Amino acids, minerals: |
| | 100 parts | 0.01 part | 4 parts |

TABLE 2

| | Starter | Grower | Finisher | Total Feed | |
|---|---|---|---|---|---|
| Test | stage | stage | stage | Consumption | |
| Group | (Term A) | (Term B) | (Term C) | (g) | Remarks |
| 1 | Feed Z1 | Feed Z1 | Feed Z1 | 3480 | Present |
| | | | | | Invention |
| 2 | Feed Z3 | Feed Z3 | Feed Z3 | 3418 | Present |
| | | | | | Invention |
| 3 | Feed Z2 | Feed N1 | Feed Z4 | 3425 | Present |
| | | | | | Invention |
| 4 | Feed N1 | Feed N1 | Feed N1 | 3458 | Control |
| 5 | Feed N2 | Feed N2 | Feed N1 | 3482 | Comparative |
| | | | | | Example |

TABLE 3

| | | Feed | | No. of | |
|---|---|---|---|---|---|
| Test | Av. body | conversion | lesion | Salmonella | |
| Group | wt. gain | ratio | score | (eval.) | Remarks |
| 1 | 1887 | 1.860 | 0.857 | 1552 (++) | Present Invention |
| 2 | 1903 | 1.813 | 0.214 | 56 (−) | Present Invention |
| 3 | 1898 | 1.819 | 0.250 | 82 (−) | Present Invention |
| 4 | 1864 | 1.870 | 1.554 | 1774 (++) | Control |
| 5 | 1887 | 1.854 | 0.393 | 167 (−) | Comparative |
| | | | | | Example |

The foregoing results clearly show that the test groups 2 and 3 show lower lesion score than that of test groups 4 (control) and 5 (Comparative group). The number of salmonella found in the results of test groups 2 and 3 are negative in the evaluation (eval.) and the meat of these chicken can be accordingly considered as highly safe products. Moreover, the test group 3 shows the intended effect without feeding the feed composition contains sliver-carrying zeolite during the grower stage and still achieved almost same effect to test group 2 which was fed the feed composition which contains silver-carrying zeolite during all the period.

### Test Example No. 2

### Test for Antibiotic Effect of Silver-Carrying Zeolite

The zeolite particles having a silver content of 2.5% by mass prepared in Preparation Example No. 1 as well as various kinds of commercially available antimicrobial agents were inspected for the antimicrobial effects on bacteria belonging to the genus Salmonella and lactobacillus which are typical useful bacteria.

The antimicrobial effects of these samples were evaluated by the determination of Minimum Inhibitory Concentration (MIC) and Minimum Bactericidal Concentration (MBC). This is a typical method approved by Japanese Society of Chemotherapy.

In this connection, bacterial strains used herein were those obtained from enteric canal of broiler chicken. The test results are listed in the following Table 4:

TABLE 4

| | |
|---|---|
| Salmonella | lactobacillus |

| Antimicrobial Agent | MIC | MBC | MIC | MBC |
|---|---|---|---|---|
| Ag-Carrying Zeolite | 125 | 62 | 1000 | 1000 |
| Silver sulfadiazine | 1000 | 500 | 500 | 125 |
| Ampicillin | >2000 | >2000 | 2000 | 1000 |

The foregoing data listed in Table 4 clearly indicate that when comparing the silver-carrying zeolite with other antimicrobial agents tested herein, the former shows a higher antibacterial effect against Salmonella, while it shows rather weak effect against lactobacillus. Incidentally, it would be recognized that Ampicillin as an antibiotics in fact shows a quite weak antibacterial effect against Salmonella since the bacteria has already acquired the resistance thereto.

As has been described above, the feed composition of the present invention has almost no influence on the useful lactobacillus, but exerts an antimicrobial effect on various kinds of harmful microorganisms, in particular, bacteria belonging to the genus Salmonella or the like. Accordingly, the feed composition of the invention would permit the improvement of the healthiness of enteric canal of domestic animals, domestic fowls and cultivated fishes such as chickens, swine, cattle, sheep, equines, carps and various kinds of cultivated fishes and the composition would in turn permit the acceleration of their growth, while maintaining their good healthiness.

What is claimed is:

1. A method for breeding an animal, wherein said method comprises:
administering a feed composition (a) to the animal in an initial breeding stage; and
administering a feed composition (b) to the animal after completion of the initial breeding stage,
wherein the feed composition (a) comprises a silver-carrying zeolite having a silver content of 0.1-5.0 wt. %, based on a total weight of the silver-carrying zeolite,
wherein the feed composition (a) comprises silver in an amount of A wt. %, based on a total weight of the feed composition (a), wherein A is 0.000001-0.2 wt. %,
wherein the feed composition (b) comprises a silver-carrying zeolite having a silver content of 0.1-5.0 wt. %, based on a total weight of the silver-carrying zeolite,
wherein the feed composition (b) comprises silver in an amount of B wt. %, based on a total weight of the feed composition (b), wherein B is less than A, and
wherein the animal is selected from the group consisting of fowl bred for egg collection and dairy cattle.

2. A method for breeding an animal, wherein said method comprises:
administering a feed composition (a) to the animal in an initial breeding stage;
administering a feed composition (b) to the animal during a term extending from after completion of the initial breeding stage to 5-20 days before a distribution or shipping date of the animal; and
administering a feed composition (c) to the animal during a term between 5-20 days before the distribution or shipping date and the distribution or shipping date of the animal,
wherein the feed composition (a) comprises a silver-carrying zeolite having a silver content of 0.1-5.0 wt. %, based on a total weight of the silver-carrying zeolite,
wherein the feed composition (a) comprises silver in an amount of A wt. %, based on a total weight of the feed composition (a), wherein A is 0.000001-0.2 wt. %,
wherein the feed composition (b) comprises a silver-carrying zeolite having a silver content of 0.1-5.0 wt. %, based on a total weight of the silver-carrying zeolite,
wherein the feed composition (b) comprises silver in an amount of B wt. %, based on a total weight of the feed composition (b), wherein B is less than A,
wherein the feed composition (c) comprises a silver-carrying zeolite having a silver content of 0.1-5.0 wt. %, based on a total weight of the silver-carrying zeolite,
wherein the feed composition (c) comprises silver in an amount of C wt. %, based on a total weight of the feed composition (c), wherein C is greater than B, and
wherein the animal is one for obtaining meat.

3. The method according to claim 2, wherein the animal is selected from the group consisting of poultry, cattle, swine, wild boar, equine, sheep and deer.

4. The method according to claim 2, wherein the animal is selected from the group consisting of domestic fowl bred for obtaining meat, beef cattle and swine.
